(19) United States
(12) Patent Application Publication
(10) Pub. No.: US 2013/0239250 A1
(43) Pub. Date: Sep. 12, 2013

(54) RESISTANCE TO NASONOVIA RIBISNIGRI BIOTYPE 1 (NR:1) FROM LACTUCA SERRIOLA

(71) Applicants: Arnaud Paul Pierre THABUIS, Montfavet (FR); Korstiaan Cornelis TEEKENS, Hoek van Holland (NL); Zeger Otto VAN HERWIJNEN, Rotterdam (NL)

(72) Inventors: Arnaud Paul Pierre THABUIS, Montfavet (FR); Korstiaan Cornelis TEEKENS, Hoek van Holland (NL); Zeger Otto VAN HERWIJNEN, Rotterdam (NL)

(73) Assignee: RIJK ZWAAN ZAADTEELT EN ZAADHANDEL B.V., DE LIER (NL)

(21) Appl. No.: 13/792,014

(22) Filed: Mar. 9, 2013

Related U.S. Application Data
(63) Continuation-in-part of application No. PCT/EP2011/070179, filed on Nov. 5, 2011.
Application No. PCT/EP2011/070179 is a continuation-in-part of application No. PCT/EP2010/067589, filed on Nov. 16, 2010.

(51) Int. Cl. A01H 5/12; A23L 1/212

(52) U.S. Cl. CPC A01H 5/12; A23L 1/212
USPC 800/265; 800/305; 435/410; 426/615

(57) ABSTRACT

The present invention relates to a lettuce plant (Lactuca sativa L.) resistant against Nasonovia ribisnigri biotype 1 (Nr:1) representative seeds of which were deposited under NCIMB accession number 41763. Such a lettuce plant may be obtainable by crossing a lettuce plant with a plant grown from seeds of seedlot 10G.913569 representative seeds of which were deposited with the NCIMB under NCIMB accession number 41763 and selecting preferably in the F2 progeny of the cross for plants being resistant against Nasonovia ribisnigri biotype 1 (Nr:1).

RELATED APPLICATIONS AND INCORPORATION BY REFERENCE

This application is a continuation-in-part application of international patent application Serial No. PCT/EP2011/070179 filed 15 Nov. 2011, which published as PCT Publication No. WO 2012/066008 on 24 May 2012, which claims benefit of international patent application Serial No. PCT/EP2010/067589 filed 16 Nov. 2010.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention relates to lettuce (Lactuca sativa L.) plants being resistant against the lettuce aphid Nasonovia ribisnigri (Mosley). The invention further relates to parts of the plants, in particular to the seeds and to other propagation material, and to progeny of the plants, which show the same resistance.

### BACKGROUND OF THE INVENTION

The lettuce aphid Nasonovia ribisnigri (Mosley) is a major pest occurring in lettuce worldwide. The problem started to be severe for lettuce production in the 70s in North Western Europe and spread rapidly all across Europe. In the 80s, the aphid was detected in Canada. Later on, the problem was reported in the USA (California and Arizona). More recently, the lettuce aphid was found in New Zealand and Australia.

Lettuce aphids can colonize lettuce plants at any plant stage and feed preferably from younger leaves. Large amounts of aphids on the plant are able to reduce plant growth and deform the shape of the head so that the lettuce heads are then not marketable. The presence of high amounts of aphids in lettuce heads is a reason for retailers to refuse to buy lettuce from growers. At young plant stage, it is possible to control the lettuce aphid using insecticide. Several products were reported efficient to control aphid population. However, resistance to chemicals were reported in some aphid populations. Moreover, at maturity, it is not possible to control aphids using insecticide because the chemical product cannot enter the lettuce head.

One of the most valuable strategies to control lettuce aphids is the genetic resistance. Extensive genebank screening was performed and some Lactuca virosa accessions were found completely resistant to Nasonovia ribisnigri. However, Lactuca virosa is in the second gene pool of the Lactuca germplasm according to the definition of Harlan. Therefore, these interspecific crosses are sterile, and the use of bridge species (as Lactuca serriola) was necessary to transfer the resistance into L. sativa. Genetic analyses showed that the resistance to Nasonovia ribisnigri was controlled by a single dominant gene (Nr gene) in a Lactuca sativa background.

However, breeders experienced that the release of varieties resistant to lettuce aphid was not straightforward. The Nr-resistance gene was found tightly linked to recessive genes conferring strong pleiotropic effect. Such plants showed a reduced growth, a pale green colour and a lack of fertility in seed set. Using large-sized progeny and molecular markers enabled Rijk Zwaan lettuce breeders to find resistant recombinant plants without the negative side-effect phenotype.

After this finding, the release of varieties resistant to Nasonovia became more and more important. The resistance trait became a major requirement for outdoor lettuce production for the processing industry and also for the fresh market. More recently, partial resistance to Nasonovia was reported in one L. serriola accession PI 491093 (Mc Creight, 2008).

In 2007, populations of lettuce aphids able to infect varieties resistant to Nasonovia ribisnigri were found in 4 distinct areas in Europe (France, Germany, Belgium and Austria). Four isolates (2 from France and 2 from Germany) were analysed further by Naktuinbouw (Netherlands Inspection Service for Horticulture) and they concluded the existence of a new Nasonovia ribisnigri biotype. This biotype is officially named Nr:1 and is able to overcome the Nr resistance gene. Nr:0 biotype can still be efficiently controlled by the Nr gene. Naktuinbouw also reported that IVT 280, the original source of the Nr:0 resistance, was susceptible in their tests.

After the breakthrough of the Nr gene by the Nr:1 biotype in practice, there was a need to find a resistance to the Nr:1 biotype.

Citation or identification of any document in this application is not an admission that such document is available as prior art to the present invention.

### SUMMARY OF THE INVENTION

In the research leading to the present invention new lettuce plants were developed which are resistant against Nr:1 as a result of a genetic determinant in their genome that causes the resistant. The resistance was surprisingly found in Lactuca serriola. L. serriola was never reported being resistant to Nasonovia ribisnigri.

The present invention thus provides a lettuce (Lactuca sativa L.) plant, which has the Nr:1 resistance as found in plants grown from seeds of seedlot 10G.913569 representative seeds of which were deposited on 29 Sep. 2010 with NCIMB Ltd, Ferguson Building, Craibstone Estate, Bucksburn, Aberdeen AB21 9YA, UK under deposit accession number NCIMB 41763.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### DEPOSIT

The Deposit with NCIMB, under deposit accession number 41763 were made pursuant to the terms of the Budapest Treaty. Upon issuance of a patent, all restrictions upon the deposit will be removed, and the deposit is intended to meet the requirements of 37 CFR §§1.801-1.809. The deposit will be maintained in the depository for a period of 30 years, or 5 years after the last request, or for the effective life of the patent, whichever is longer, and will be replaced if necessary during that period.

### DETAILED DESCRIPTION OF THE INVENTION

The present invention thus provides a lettuce (Lactuca sativa L.) plant, which has the Nr:1 resistance as found in plants grown from seeds of seedlot 10G.913569 representative seeds of which were deposited under deposit accession number NCIMB 41763.

In one embodiment the invention thus relates to a lettuce plant (Lactuca sativa L.) having in its genome a genetic determinant that confers resistance against Nasonovia ribisnigri biotype 1 (Nr:1), which genetic determinant is as present in seeds of which a representative sample was deposited under NCIMB accession number 41763.

The invention also relates to the use of L. serriola as a source of resistance against Nasonovia ribisnigri biotype 1 (Nr:1). The genetic determinant that causes the resistance and may thus be considered as the source of the resistance is suitably introduced in susceptible lettuce plants (L. sativa) by introgression.

The invention further relates to Lactuca sativa plants that are resistant against Nasonovia ribisnigri biotype 1 (Nr:1), obtainable by crossing a L. serriola plant with a Lactuca sativa plant.

According to a further aspect thereof, the invention provides progeny of the plant as claimed, seeds of the plant as claimed and parts of the plant as claimed.

In one embodiment, the invention relates to a lettuce plant resistant against Nr:1, wherein said resistance is obtainable by introgression from a plant of which representative seed was deposited with the NCIMB under accession number NCIMB 41763.

In one embodiment, the invention relates to a lettuce plant resistant against Nr:1, wherein said trait is introgressed from a plant of which representative seed was deposited with the NCIMB under accession number NCIMB 41763. “Introgression” as used herein is intended to mean introduction of a Nr:1 resistance into a plant not carrying the Nr:1 resistance by means of crossing and selection. Introgression is not limited to one cross only but encompasses the minimum number of generations needed for the plant to become resistant, preferably stably resistant.

In one embodiment the invention relates to a lettuce plant resistant against Nr:1, obtainable by crossing a lettuce plant susceptible to Nr:1 with a plant of which representative seeds were deposited with the NCIMB under NCIMB accession number 41763 and selecting preferably in the F2 progeny of the cross for plants being resistant against Nasonovia ribisnigri biotype 1 (Nr:1). In one embodiment, the invention relates to a lettuce plant resistant against Nr:1, which plant is obtainable by crossing a lettuce plant with a plant grown from seedlot 10G.913569 representative seeds of which were deposited with the NCIMB under NCIMB accession number 41763 and selecting in the F2 progeny of the cross, which is obtained after selfing the F1, for plants being resistant against Nasonovia ribisnigri biotype 1 (Nr:1).

In one embodiment the invention provides a lettuce plant having in its genome a genetic determinant that confers resistance against Nasonovia ribisnigri biotype 1 (Nr:1), which plant is obtainable by crossing a plant grown from seeds of seed lot 10G.913569, representative seeds of which were deposited with the NCIMB under NCIMB accession number 41763, with another plant and selecting preferably in the F2 progeny of the cross for plants being resistant against Nasonovia ribisnigri biotype 1 (Nr:1).

In one embodiment the invention provides a lettuce plant, being resistant against Nr:1 as compared to a susceptible control plant and having in its genome a genetic determinant that confers resistance against Nasonovia ribisnigri biotype 1 (Nr:1), which plant is obtainable by crossing a lettuce plant with a plant grown from seeds of seed lot 10G.913569, representative seeds of which were deposited with the NCIMB under NCIMB accession number 41763, and selecting in the F2 progeny of the cross that is obtained after selfing the F1 for plants being resistant against Nr:1.

The resistance trait or genetic determinant as present in the genome of the deposited material is transferred in a pattern consistent with recessive inheritance, and selection is thus suitably made in the second generation (F2) after having selfed the F1.

In one embodiment, the invention further relates to a lettuce plant resistant against Nr:1, wherein when said plant is homozygous for said trait and said plant homozygous for said trait is crossed with a tester plant homozygous for the said trait, plants of the first generation progeny resulting from said cross show a 1:0 segregation for resistance against Nr:1.

In one embodiment, when said plants of said first generation progeny are self-pollinated, plants of the resulting second generation progeny show a 1:0 segregation for resistance against Nr:1. In one embodiment, the tester plant is a lettuce plant grown from seeds deposited with the NCIMB under NCIMB accession number 41763.

In one embodiment, the tester plant is a plant grown from seeds deposited with the NCIMB under NCIMB accession number 41763, or a progeny of said plant which is resistant against Nr:1.

In one embodiment, the lettuce plants of the invention do essentially not show any negative pleiotropic effects.

The invention further relates to seed of the lettuce plants of the invention and to other parts of the plant that are suitable for sexual reproduction, i.e. propagation material. Such parts are for example selected from the group consisting of microspores, pollen, ovaries, ovules, embryo sacs and egg cells.

In one embodiment seeds of a lettuce plant are provided, wherein a lettuce plant that is grown from the seed is resistant against Nr:1 as a result of the presence in its genome of a genetic determinant that confers resistance against Nasonovia ribisnigri biotype 1 (Nr:1), which determinant is as present in seeds having NCIMB accession number 41763.

In one embodiment, propagation material of a plant of the invention is provided, which is suitable for sexual reproduction, in particular microspores, pollen, ovaries, ovules, embryo sacs and egg cells, wherein the propagation material carries the genetic trait that confers resistance against Nasonovia ribisnigri biotype 1 (Nr:1) as present in seeds having NCIMB accession number 41763. In addition, the invention relates to parts of the plant that are suitable for vegetative reproduction, in particular cuttings, roots, stems, cells, protoplasts, and tissue culture of the lettuce plants of the invention. The tissue culture may comprise regenerable cells. Such a tissue culture may be derived from leaves, pollen, embryos, cotyledon, hypocotyls, meristematic cells, roots, root tips, anthers, flowers, seeds and stems.

In one embodiment, plant parts of a plant of the invention are provided, which are suitable for vegetative reproduction, in particular cuttings, roots, stems, cells, protoplasts, and tissue culture, wherein the propagation material carries the genetic trait that confers resistance against Nasonovia ribisnigri biotype 1 (Nr:1) as found in seeds having NCIMB accession number 41763.

The invention also relates to progeny of the lettuce plants of the invention. Such progeny may be produced by sexual or vegetative reproduction of a plant of the invention or a progeny plant thereof.

The regenerated progeny plants have the same genetic basis for resistance against Nr:1 as the plant of which representative seed was deposited under NCIMB accession number NCIMB 41763. This means that in case the genetic basis is homozygous such progeny has the same characteristics as claimed for the lettuce plants of the invention. In case the genetic basis is present in heterozygous form, the progeny does not show resistance against Nr:1 as is present in the plant of the invention, but still contains the alleles responsible for the Nr:1 resistance, and is therefore still a source for Nr:1 resistance.

In one embodiment, progeny of a plant or seed of the invention is provided, which progeny is resistant against Nr:1 as a result of the presence in its genome of a genetic trait that confers resistance against Nasonovia ribisnigri biotype 1 (Nr:1) as present in seeds having NCIMB accession number 41763.

In addition to this, the plant may be modified in one or more other characteristics besides the already present Nr:1 resistance. Such additional modifications are for example effected by mutagenesis or by transformation with a transgene. Alternatively, modifications in characteristics other than the Nr:1 resistance may be introduced by introducing the Nr:1 resistance in a different background. In the first case the resistant plant is taken as the original plant and modified. In the latter case the resistance is transferred to another plant, in particular a susceptible plant. Modification of other traits may also occur through further breeding.

As used herein the word “progeny” is intended to mean the offspring or the first and all further descendants from a cross with a plant of the invention that is Nr:1 resistant. Progeny of the invention are descendants of any cross with a plant of the invention that carries the Nr:1 resistance, in particular as caused by the genetic determinant that is present in the genome of the deposited seeds with NCIMB accession number 41763. Progeny plants are preferably also resistant.

“Progeny” also encompasses plants that carry the Nr:1 resistance of the invention which are obtained from other plants of the invention by vegetative propagation or multiplication.

In one embodiment, the invention relates to lettuce plants that carry the trait of the invention, being resistant against Nr:1, and having acquired said trait by introduction of the genetic information that is responsible for the trait (i.e. the genetic determinant) from a suitable source, either by conventional breeding, or genetic modification, in particular by cisgenesis or transgenesis. Cisgenesis is genetic modification of plants with a natural gene, coding for an (agricultural) trait, from the crop plant itself or from a sexually compatible donor plant. Transgenesis is genetic modification of a plant with a gene from a non-crossable species or a synthetic gene.

In one embodiment, the source from which the genetic information or determinant is acquired is formed by plants grown from the deposited seeds or sexual or vegetative descendants therefrom.

The invention, furthermore, relates to hybrid seed and to a method of producing hybrid seed which may comprise crossing a first parent plant with a second parent plant and harvesting the resultant hybrid seed. In order for the hybrid seed to express the Nr:1 resistance of the invention, at least one of the parent plants needs to be homozygous for the Nr:1 resistance but is not necessarily uniform for other characteristics.

The invention also relates to the germplasm of plants of the invention. The germplasm is constituted by all inherited characteristics of an organism and according to the invention encompasses at least the genetic determinant that causes the trait of the invention, i.e. the resistance against Nr:1.

The invention further relates to cells of the lettuce plants are resistant against Nr:1. Each cell of such lettuce plants carries the genetic information or genetic determinant that leads to phenotypic expression of said trait. The cell may be an individual cell or be part of a lettuce plant or lettuce plant part.

The invention further relates to the head of an Nr:1 resistant lettuce plant as claimed.

In one embodiment, the invention provides a head of a lettuce plant as claimed in any one of the claims 1-5, which lettuce plant is resistant against Nasonovia ribisnigri biotype 1 (Nr:1) as a result of the presence in its genome of a genetic determinant that confers resistance against Nasonovia ribisnigri biotype 1 (Nr:1), which determinant is as present in seeds having NCIMB accession number 41763.

The invention further relates to a food product, which may comprise the Nr:1 resistant lettuce head or parts thereof. In particular, the food product may comprise leaves of the Nr:1 resistant lettuce plant or parts thereof. The food product is for example a salad or a salad mixture which may comprise leaves of the lettuce plant of the invention.

In this specification the term “Nr:1 resistance” is intended to mean having the genetic information (in particular a gene, genes, locus, loci, allele or alleles) that in homozygous state leads to the plant no longer being susceptible to Nasonovia ribisnigri biotype 1 (Nr:1) and which resistance is as found in the genome of plants of which representative seeds were deposited under accession number NCIMB 41763, in particular in plants grown from the deposited seeds. The genetic determinant that is present in the genome of plants of the invention is the same or similar to the genetic determinant that is the cause of the resistance in plants grown from the deposited seeds with NCIMB accession number 41763.

The term “genetic determinant” is intended to mean any gene, locus or allele or set of genes, loci or alleles that causes the phenotypic expression of resistance to Nasonovia ribisnigri biotype 1 (Nr:1) and thus underlies the resistance phenotype.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLE

### Protocol of the Young-Plant Test

In order to test whether a plant is resistant to Nr:1 the following test is performed.

Seeds are sown in potting compost blocks. Twenty plants per line were evaluated. The young plants are inoculated 2 weeks after sowing by pouring aphids of the Nr:1 biotype on the top of the plants. One week after inoculation, the test is scored. The second and final score occurs two weeks after inoculation.

### Nasonovia ribisnigri Isolates

The Nr:1 isolate used in this example was sampled in 2007 from Köningen (Germany) on a Nr:0 resistant variety Estelle (Nunhems): sample 279. The aphid isolate is maintained on the variety Kitare (Nr:0 resistant variety, Rijk Zwaan).

### Introduction of the Nr:1 Resistance into Susceptible Lettuce

A cross between a susceptible lettuce variety L. sativa used as a female and L. serriola used as a male was performed. The F1 seeds were sown and the F2 generation was obtained. After this, 150 F2 seeds were sown and propagated to generate 150 F3 lines. From these, 100 F3 lines were included in Nr:0 and Nr:1 tests.

The F3 line 09D.857022 was found resistant in 2008 (in test 08D.846) and the results were confirmed in 2009 (in test 09D.846). From the test 08D.846, 3 resistant plants were selected. This F3 line was also tested against Nr:0 twice and this line displayed a susceptible reaction. The 3 F4 lines resulting from the 3 resistant F3 plants (09D.861266, 09D.861267 and 09D.861268) were then tested against Nr:1 in test 09D.841 and the 3 lines displayed a resistant behaviour. From 09D.861266, 4 F5 plants were selected. Those 4 F5 lines (09D.873417, 09D.873418, 09D.873419 and 09D.873420) were then included in test 09D.846 and all of them displayed a resistant reaction. From line 09D.874317, 10 plants were selected from this test. The F6 lines were tested in 10D.831 and all the lines were detected resistant. The 10 lines were bulked to obtain a seed lot for a seed deposit, 10G.913569.

Nr:1 resistant L. sativa 10G.913569 plants (called herein “the donors”) were used to introgress the Nr:1 resistance into susceptible lettuce varieties.

The donors were crossed to a cultivated susceptible lettuce variety. Cultivated varieties belonging to different lettuce types were used. The F1s obtained were selfed. Then, the different F2 populations were placed in a Nr:1 disease resistance test as described above. At the end of the test, Nr:1 resistant plants were selected for further development.

The invention is further described by the following numbered paragraphs:

1. A lettuce plant (Lactuca sativa L.) having in its genome a genetic determinant that confers resistance against Nasonovia ribisnigri biotype 1 (Nr:1), which genetic determinant is as present in seeds of which a representative sample was deposited under NCIMB accession number 41763.

2. Lettuce plant of paragraph 1 having in its genome a genetic determinant that confers resistance against Nasonovia ribisnigri biotype 1 (Nr:1), which plant is obtainable by crossing a plant grown from seeds of seed lot 10G.913569, representative seeds of which were deposited with the NCIMB under NCIMB accession number 41763, with another plant and selecting preferably in the F2 progeny of the cross for plants being resistant against Nasonovia ribisnigri biotype 1 (Nr:1).

3. Lettuce plant of paragraph 1 or 2, being resistant against Nr:1 as compared to a susceptible control plant and having in its genome a genetic determinant that confers resistance against Nasonovia ribisnigri biotype 1 (Nr:1), which plant is obtainable by crossing a lettuce plant with a plant grown from seeds of seed lot 10G.913569, representative seeds of which were deposited with the NCIMB under NCIMB accession number 41763, and selecting in the F2 progeny of the cross that is obtained after selfing the F1 for plants being resistant against Nr:1.

4. Seed of a lettuce plant of any one of the paragraphs 1-3, wherein a lettuce plant that is grown from the seed is resistant against Nr:1 as a result of the presence in its genome of a genetic determinant that confers resistance against Nasonovia ribisnigri biotype 1 (Nr:1), which determinant is as present in seeds having NCIMB accession number 41763.

5. Progeny of a plant or seed of any of the paragraphs 1-4, which progeny is resistant against Nr:1 as a result of the presence in its genome of a genetic trait that confers resistance against Nasonovia ribisnigri biotype 1 (Nr:1) as present in seeds having NCIMB accession number 41763.

6. Propagation material of a plant of any one of the paragraphs 1 to 5, suitable for sexual reproduction, in particular microspores, pollen, ovaries, ovules, embryo sacs and egg cells, wherein the propagation material carries the genetic trait that confers resistance against Nasonovia ribisnigri biotype 1 (Nr:1) as present in seeds having NCIMB accession number 41763.

7. Plant parts of a plant of any one of the paragraphs 1 to 5, suitable for vegetative reproduction, in particular cuttings, roots, stems, cells, protoplasts, and tissue culture, wherein the propagation material carries the genetic trait that confers resistance against Nasonovia ribisnigri biotype 1 (Nr:1) as found in seeds having NCIMB accession number 41763.

8. Head of a lettuce plant of any one of the paragraphs 1-5, which lettuce plant is resistant against Nasonovia ribisnigri biotype 1 (Nr:1) as a result of the presence in its genome of a genetic determinant that confers resistance against Nasonovia ribisnigri biotype 1 (Nr:1), which determinant is as present in seeds having NCIMB accession number 41763.

9. Food product, comprising the lettuce head of paragraph 8 or parts thereof.

10. Food product of paragraph 8, wherein the parts are leaves or parts thereof.

11. Use of Lactuca sativa 10G.913569, representative seeds of which were deposited under NCIMB accession number 41763, in breeding to confer resistance against Nasonovia ribisnigri biotype 1 (Nr:1) to plants that are susceptible to Nasonovia ribisnigri biotype 1 (Nr:1).

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A lettuce plant (Lactuca sativa L.) having in its genome a genetic determinant that confers resistance against Nasonovia ribisnigri biotype 1 (Nr:1), which genetic determinant is as present in seeds of which a representative sample was deposited under NCIMB accession number 41763.

2. A lettuce plant as claimed in claim 1 having in its genome a genetic determinant that confers resistance against Nasonovia ribisnigri biotype 1 (Nr:1), which plant is obtainable by crossing a plant grown from seeds of seed lot 10G.913569, representative seeds of which were deposited with the NCIMB under NCIMB accession number 41763, with another plant and selecting preferably in the F2 progeny of the cross for plants being resistant against Nasonovia ribisnigri biotype 1 (Nr:1).

3. A lettuce plant as claimed in claim 1, being resistant against Nr:1 as compared to a susceptible control plant and having in its genome a genetic determinant that confers resistance against Nasonovia ribisnigri biotype 1 (Nr:1), which plant is obtainable by crossing a lettuce plant with a plant grown from seeds of seed lot 10G.913569, representative seeds of which were deposited with the NCIMB under NCIMB accession number 41763, and selecting in the F2 progeny of the cross that is obtained after selfing the F1 for plants being resistant against Nr:1.

4. A seed of a lettuce plant as claimed in claim 1, wherein a lettuce plant that is grown from the seed is resistant against Nr:1 as a result of the presence in its genome of a genetic determinant that confers resistance against Nasonovia ribisnigri biotype 1 (Nr:1), which determinant is as present in seeds having NCIMB accession number 41763.

5. A progeny of a plant or seed as claimed in claim 1, which progeny is resistant against Nr:1 as a result of the presence in its genome of a genetic trait that confers resistance against Nasonovia ribisnigri biotype 1 (Nr:1) as present in seeds having NCIMB accession number 41763.

6. A propagation material of a plant as claimed in claim 1, suitable for sexual reproduction, in particular microspores, pollen, ovaries, ovules, embryo sacs and egg cells, wherein the propagation material carries the genetic trait that confers resistance against Nasonovia ribisnigri biotype 1 (Nr:1) as present in seeds having NCIMB accession number 41763.

7. Plant parts of a plant as claimed in claim 1, suitable for vegetative reproduction, in particular cuttings, roots, stems, cells, protoplasts, and tissue culture, wherein the propagation material carries the genetic trait that confers resistance against Nasonovia ribisnigri biotype 1 (Nr:1) as found in seeds having NCIMB accession number 41763.

8. A head of a lettuce plant as claimed in claim 1, which lettuce plant is resistant against Nasonovia ribisnigri biotype 1 (Nr:1) as a result of the presence in its genome of a genetic determinant that confers resistance against Nasonovia ribisnigri biotype 1 (Nr:1), which determinant is as present in seeds having NCIMB accession number 41763.

9. A food product, comprising the head of a lettuce plant of claim 8 or parts thereof.

10. The food product as claimed in claim 9, wherein the parts are leaves or parts thereof.

11. A method of conferring resistance against Nasonovia ribisnigri biotype 1 (Nr:1) to plants that are susceptible to Nasonovia ribisnigri biotype 1 (Nr:1) comprising breeding Lactuca sativa 10G.913569, representative seeds of which were deposited under NCIMB accession number 41763, to the plants that are susceptible to Nasonovia ribisnigri biotype 1 (Nr:1), thereby conferring resistance against Nasonovia ribisnigri biotype 1 (Nr:1) to plants that are susceptible to Nasonovia ribisnigri biotype 1 (Nr:1).
